(19) United States
(12) Patent Application Publication
(10) Pub. No.: US 2019/0228868 A1
(43) Pub. Date: Jul. 25, 2019

(54) SYSTEM AND METHODS FOR MATCHING PROFESSIONALS WITH CLIENTS

(71) Applicant: STRUAN COLEMAN, NEW YORK, NY (US)

(72) Inventor: STRUAN COLEMAN, NEW YORK, NY (US)

(21) Appl. No.: 16/332,936

(22) PCT Filed: Sep. 21, 2017
(86) PCT No.: PCT/US17/52656

§ 371 Date: Mar. 13, 2019

Related U.S. Application Data
(60) Provisional application No. 62/397,496, filed on Sep. 21, 2016.

(51) Int. Cl. G16H 80/00; G16H 10/60; G06Q 10/10

(52) U.S. Cl. CPC G16H 80/00; G06Q 10/1095; G16H 10/60

(57) ABSTRACT

The present invention relates to methods and systems for connecting doctors with patients. More particularly, the present invention relates to computer implemented methods and systems that permit doctors to screen a plurality of patients based on preselected filter criteria.

### FIELD OF INVENTION

The present invention relates to methods and systems for connecting doctors with patients. More particularly, the present invention relates to computer implemented methods and systems that permit doctors to screen a plurality of patients based on preselected filter criteria.

### BACKGROUND

Professional services from certified and/or trained professionals (e.g., doctors, lawyers, accountants, fiduciaries, etc.) are routinely required by clients wishing to patronize their services. However, rapidly expanding knowledge and techniques of these professionals has resulted in increasing sub-specialization within professional service fields. When a client requires a more specialized focus from a trained professional in a field, it is often necessary to obtain referrals from other and oftentimes less specialized professionals within that field. Additionally, the ease of access of information and databases has provided potential clients the ability to search through practitioners of these sub-specialties to select professionals that the client believes will be able to serve their need. These searches are typically based only on criteria the client chooses and does not offer a full scope of the client's desires. For example, if a patient is searching for a specific type of medical doctor, the patient may search for doctors with specific specialties, for doctors that accept payment from the patient's medical insurance carrier, and/or for doctors in a geographic location proximal to the patient. This often results in a lengthy number of potential matches for the client without any way to differentiate professionals. Additionally, clients seeking on their own to find professionals often suffer from consumer confusion with regard to terminology and protocol, and thus have a difficult time identifying the appropriate professionals.

Due, in part, to the potential asymmetry in knowledge bases between clients and professionals, clients often find, are referred to, or schedule meetings with a professional that does not ultimately meet the client's needs. When this occurs, the client must go through the process of meeting the professional and discussing the services they require, only to find out that the professional is unable to offer them the specific professional service required. Even though the professional may be able to quickly gauge their ability to help a client based on the information and desires of the client, the professional may not obtain this information until after one or more meetings have occurred. This is also inefficient for the doctor as they may spend significant time processing and meeting potential patients that ultimately do not engage their services because for example, insurance coverage of the patient. It is therefore an object of this invention to provide methods and systems to streamline the process of providing professional services, particularly specialized professional services, to clients.

The foregoing discussion is presented solely to provide a better understanding of the nature of the problems confronting the art and should not be construed in any way as an admission as to prior art, nor should the citation of any reference herein be construed as an admission that such reference constitutes “prior art” to the instant application.

### SUMMARY OF INVENTION

In accordance with one or more of the foregoing objectives and others, the present invention provides methods and systems for arranging meetings between professionals and clients. In the following description, references are made to doctors, patients, and terms relating to the practice of medicine, although it will be understood that the systems and methods described herein are equally applicable to any professional/client relationship to which a client may search for.

The present invention provides for methods and systems of connecting a potential patient with a doctor that may match patient medical profiles of patients with doctors. In the present invention, doctors may be given the ability to choose patients based on medical information presented to them before appointments are scheduled. In some embodiments, the methods are performed over a computer network or within a computer. In some embodiments, the present invention provides methods of connecting a potential patient with a doctor which may comprise:
a. receiving patient information from a user including an identification of a medical need;
b. receiving doctor information and filter criteria from a doctor, where said doctor information may comprise contact information for said doctor and/or said doctor's practice;
c. comparing patient information with filter criteria to determine if a match exists;
d. transmitting to the doctor the patient information if a match exists;
e. receiving a decision from the doctor indicating whether the doctor is willing to schedule an appointment with the user; and
f. transmitting to the user, said contact information.

The present invention may offer doctors the ability to quickly streamline the selection of potential patients. In some embodiments, the method may comprise repeating steps (a)-(h) with at least one other potential patient. In various embodiments, the method may comprise transmitting patient information that matches the filter criteria from another user after receiving a decision from the doctor.

The present invention also may offer users the ability to only search one or more doctors that are willing to schedule an appointment with them. In some embodiments, the method may comprise receiving at least two (e.g. three, one hundred, one thousand, ten thousand, etc.) sets of doctor information and filter information from a plurality of doctors. In some embodiments, the steps (a)-(h) are performed with a plurality of doctors each with independent doctor information and filter criteria. In the case of a plurality of doctors willing to schedule an appointment with a patient, the method may further comprise transmitting the sets of doctor information of the plurality of doctors willing to schedule an appointment to a user.

The method may also facilitate making the appointment between patient and doctor. In some embodiments, the method may comprise receiving available appointment times from a doctor once they have indicated a willingness to schedule an appointment. In some embodiments, the method may comprise scheduling an appointment. In some embodiments, the user may be able to transmit filter criteria so that patient information is sent only to doctors that match one or attributes of the doctors indicated by the filter criteria (e.g., specific locations of doctors, ratings of doctors, number of awards, language spoken by doctor, etc.). In some embodiments, the method may comprise transmitting to the user the doctor information. In some embodiments, the transmission of doctor information may occur after the decision from the doctor is received. In some embodiments, the method may comprise receiving from the user a selection indicating the patient is willing to see the doctor.

The method may also comprise the ability to refer patient information to other doctors. In some embodiments, the method comprises receiving referral information for one or more other practitioners from said doctor if the doctor is unwilling to schedule an appointment.

The method may also comprise transmitting additional information (e.g., doctor ratings, descriptions of available services, amenities close to the doctor, etc.) to the user. In some embodiments, the method may comprise transmitting to the user information specific to the geographic location proximal with the practice location of the doctor. In some embodiments, the information may be advertising. In some embodiments, the advertising may relate to amenities. In other embodiments, the type of advertising may be selected from the group consisting of dining, food delivery, transportation, entertainment, pharmacies nursing services, rehabilitation services, etc. In some embodiments, the information may be advertising specific to the geographic location of the doctor's practice location. In some embodiments, the method may comprise transmitting patient evaluations to a doctor. Patient evaluations may be collected from previous patients of the doctor. In some embodiments, patients may transmit an evaluation of the doctor following their appointment time. In various embodiments, the patient evaluations may comprise a rating system which may be, for example, numeric. By “numeric,” it is meant that a number may be associated with the quality of the doctor where high (or low) numbers may indicate a better patient experience. The numbers may be expressed as, for example, numbers, stars, hearts, etc. In various embodiments, the doctor information may be data about a doctor's experience, credentials, schooling, and/or awards.

In some embodiments, the software application may comprise a user interface component for selecting specialties of care with visual representations. The visual representations may be visual representations of specialties of care, types of pain, locations of pain, or combinations thereof.

### BRIEF DESCRIPTION OF THE FIGURES

FIG. 1 is a flow chart representation of an embodiment of the invention.

FIG. 2 is a flow chart representation of another embodiment of the invention.

FIG. 3 is a flow chart representation of another embodiment of the invention.

FIG. 4 is a flow chart representation of another embodiment of the invention.

FIG. 5 is a flow chart representation of another embodiment of the invention.

FIG. 6 is a graphical user interface for inputting patient information of another embodiment of the invention.

FIG. 7 is a graphical user interface for inputting doctor information and filter criteria of another embodiment of the invention.

FIG. 8a is a graphical user interface for selecting patients of another embodiment of the invention.

FIG. 8b is a graphical user interface for selecting patients of another embodiment of the invention.

FIG. 9 is a graphical user interface for selecting doctors of another embodiment of the invention.

### DETAILED DESCRIPTION OF INVENTION

All terms used herein are intended to have their ordinary meaning unless otherwise provided.

Detailed embodiments of the present invention are disclosed herein; it is to be understood that the disclosed embodiments are merely illustrative of the invention that may be embodied in various forms. In addition, each of embodiments depicted are intended to illustrative, and not restrictive. Therefore, specific structural and functional details disclosed herein are not to be interpreted as limiting, but merely as a representative basis for teaching one skilled in the art to employ the present invention.

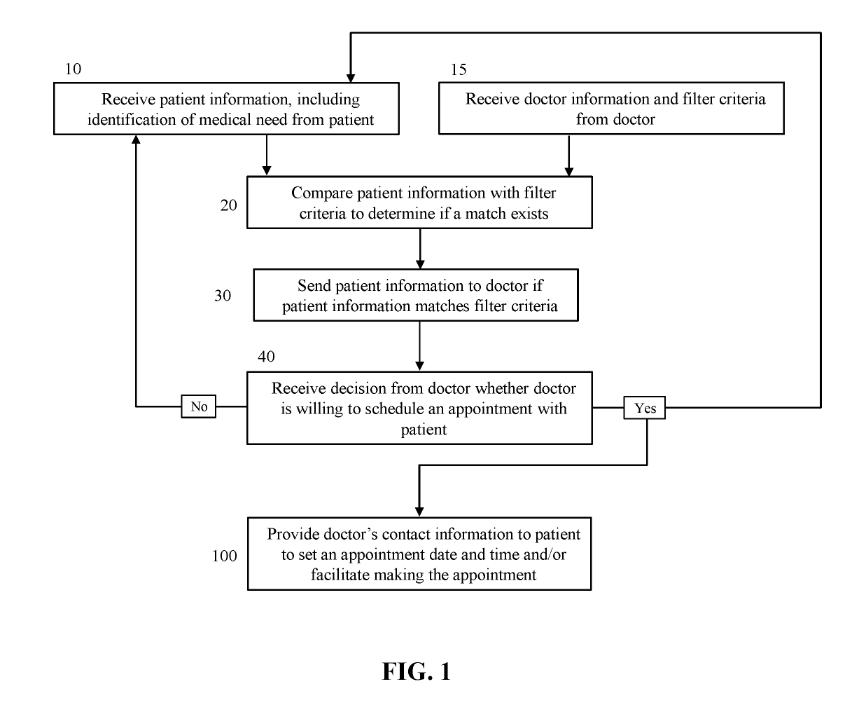

Some embodiments of the invention may comprise a method and system of connecting a potential patient with a doctor. Referring now to FIG. 1, the flow chart depicts an embodiment of the invention comprising a method of connecting a patient with patient information to a doctor. In step 10, patient information about a patient is received from a user which includes the identification of a medical need. The user may be a patient, a patient's agent, a guardian, a referring doctor, an insurance company, a patient group, a government agency or the like. The patient information may comprise the identification of a medical need, insurance information of the patient, languages spoken, willingness to find a doctor out of insurance network, medical diagnoses, results and/or data from medical tests (e.g. x-rays, MRI, CT scan, blood tests, etc.), prescriptions taken, medical records, health assessments, lifestyle considerations (e.g., smoking, eating, or exercise habits, etc.) or combinations thereof. In step 15 doctor information and filter criteria are received from a doctor. In some embodiments, the doctor information is transmitted by a doctor, a doctor's agent, a nurse, an assistant, a health care provider group, etc. The doctor information may be, for example, specialties of the doctor, number and type of surgeries performed, schools attended by the doctor, awards received by the doctor etc. In some embodiments, the doctor information comprises contact information for the doctor and/or for the doctor's practice. In some embodiments, the doctor information comprises available appointment times. The doctor information and filter criteria may be received at the same time or at different times. For example, doctor information without filter criteria may have been received at a point in time, and filter criteria is received at a later point in time. In some embodiments, doctor information is stored in an accessible database and once the filter criteria is received from the doctor, the method may be initiated.

Following steps 10 and 15, the patient information and filter criteria are compared at step 20. The filter criteria may allow the doctor to select the features of patients they may be willing to look at. In some embodiments, the doctor may be willing to only look at patient information for patients in a certain insurance network. In some embodiments, the doctor may be willing to look at patient information for patients with a certain insurance plan. In some embodiments, the doctor may be willing to look at patients with a particular disease or affliction. In some embodiments, the doctor may be willing to see only patients that do not plan to use insurance for payment. In some embodiments, the doctor may be willing to see only patients that have a particular lifestyle. If the patient information matches the filter criteria, the patient information is sent to the doctor at step 30. In some embodiments, the patient information excluding the identity of the patient is transmitted to the doctor. The doctor is then able to decide whether they are willing to schedule an appointment with the patient. Their decision is received at step 40. Following input from the doctor regarding their willingness to see a patient, and regardless of the actual decision, the doctor has the option of continuing to browse the patient information of other patients that matches the filter criteria set by the doctor. In this case, step 40 is part of an iterative process with different sets of patient information supplied at step 10. If the doctor has indicated a willingness to see the patient, the doctor's contact information is sent to the patient to make an appointment date and time and/or the appointment is made at step 100. In some embodiments, the contact information is sent to the user. In some embodiments, the contact information is sent to the patient. In some embodiments the contact information is sent to the user and the patient. In some embodiments, the contact information is sent to a third party. In some embodiments, the contact information is transmitted by the doctor. In some embodiments, the contact information is gathered from available data (e.g. the doctor's website, insurance company database, hospital database, integrated health network website, etc).

In some embodiments, patient filter criteria may be received from the user which dictates which doctors the patient information is transmit to at step 30. In some embodiments, patient filter criteria be received which dictates which doctor information or contact information is transmitted to the user and/or patient at step 100. The filter criteria may be, for example, doctors in one or more locations, doctors with a rating between a range set, doctors with a number of awards, doctors that have performed a number of surgeries, etc. In some embodiments, the patient information is sent to the doctor if the patient information matches the filter criteria and the doctor information matches the patient filter criteria.

In some embodiments, the methods and systems operate on a computer network. In some embodiments, the methods and systems operate over a computer network, a cellular network, or the like, over direct input at a local computer, or combinations thereof. In some embodiments, transmission and receipt of patients and/or doctor information occurs over direct input at a local computer. In some embodiments, the computer network may comprise one or more servers designed to receive and transmit information. In some embodiments, the doctors and patients are able to send and receive information to and from the server, respectively through a device configured to interact with the server (i.e. a client). In some embodiments, the device may be, for example, a laptop, computer, smart telephone, cellular telephone, telephone, pager, or tablet.

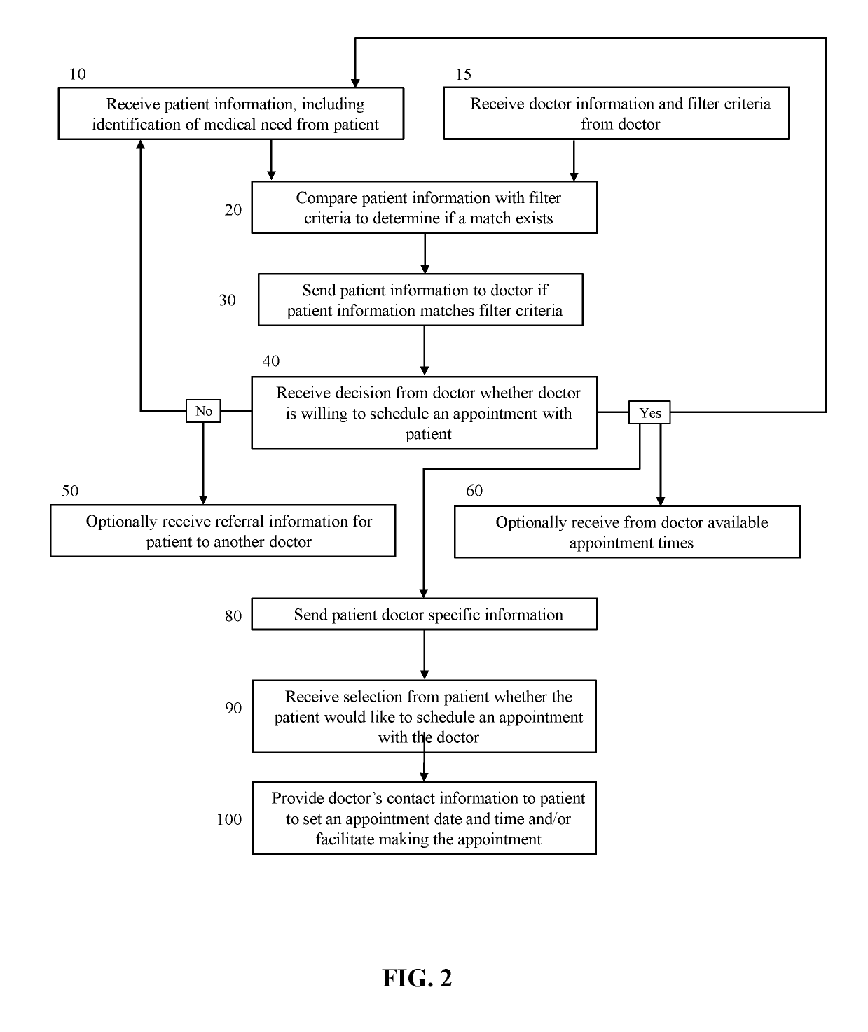

Referring now to FIG. 2, another embodiment is depicted comprising steps 10, 15, 20, 30, 40 and 100. Following the decision at step 40, if the doctor indicates an unwillingness to see a patient, the doctor has the option to offer a referral to another practitioner who the doctor believes may be willing to see the patient. Depending on the filter criteria set by the other practitioner (i.e., the filter criteria for the other practitioner may be set to include or exclude referrals), the patient's information may be presented to the other practitioner in one of the embodiments as described herein, through informing the patient of the referral, informing the doctor of the referral, or any combination thereof. The referral information is received from the doctor at step 50. If the doctor indicates a willingness to see the patient, the doctor may additionally send the available appointment times that the doctor is willing to meet with the patient during. The available appointment times are received at step 60. After a decision on the doctor's willingness to have an appointment with the patient is received, the patient is sent the doctor specific information at step 80. The doctor specific information may include or exclude the identity of the doctor. The patient then selects whether the patient would like to make an appointment with the doctor. Receipt of the selection occurs at step 90. In this embodiment, to facilitate making the appointment, if the available appointment times are supplied by the doctor, a selection of the desired appointment time for the patient may be received from the user.

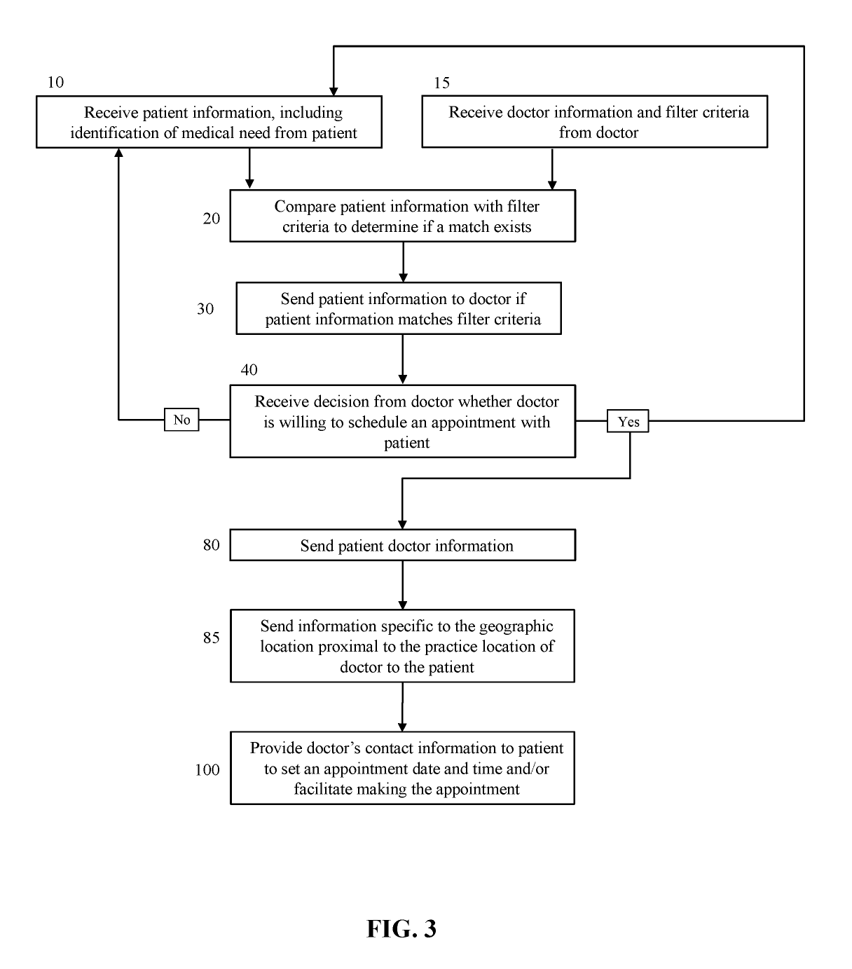

Referring now to FIG. 3, a method is depicted comprising the transmission of additional information (e.g. information about the location proximal to the practice location, information about locations in other areas from the practice location) to the patient in step 85. If the doctor has indicated a willingness to make an appointment with the patient at step 40, the doctor information is sent to the patient at step 80. In this embodiment, the patient is sent information specific to the geographic location proximal to the practice location of the doctor at step 85. This information may be, for example, advertising, reviews of particular destinations located in the location proximal to the doctor's practice, etc. In some embodiments, this advertising is focused on dining, food delivery, transportation, entertainment, pharmacies, nursing services, rehabilitation services, other doctors in the area, insurance carriers, banking or financial services, medical services in different locations from the location of the willing doctor, etc. In this embodiment, step 85 is depicted as occurring after step 80. In some embodiments, step 85 occurs simultaneously, before, or after step 80.

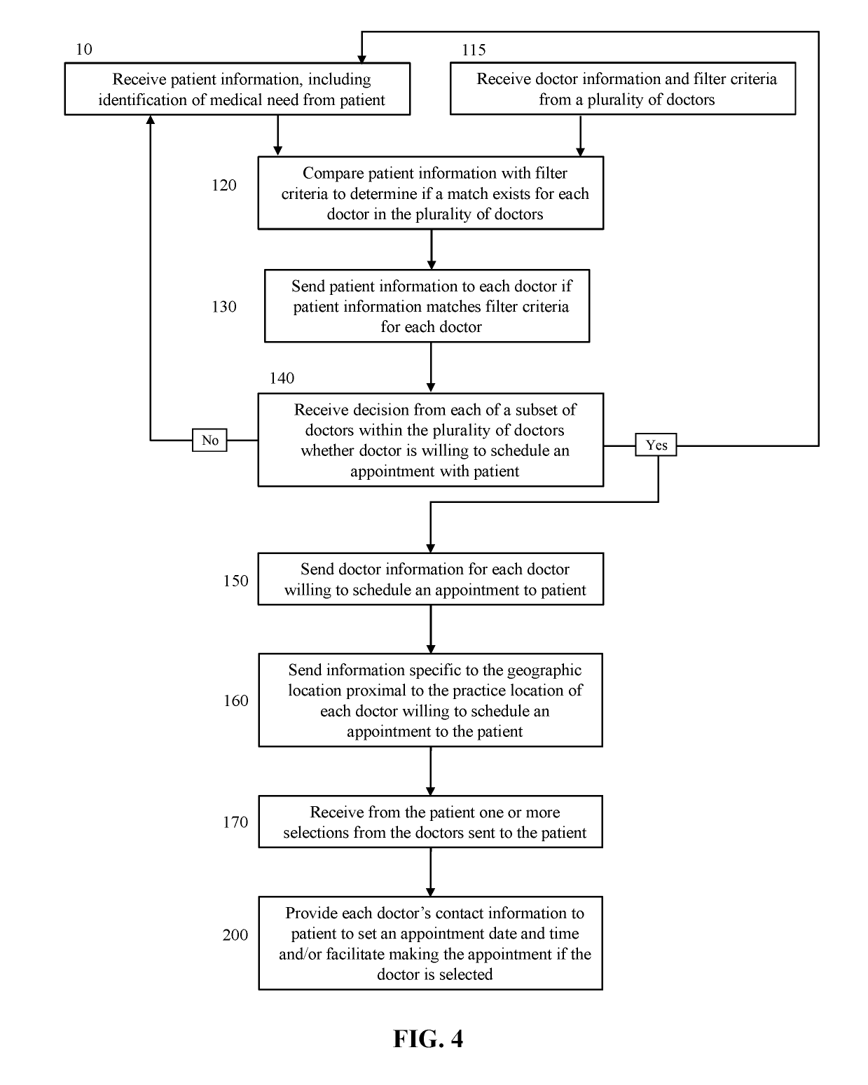

Referring now to FIG. 4, doctor information and filter criteria from a plurality of doctors is received at step 115. Each doctor within the plurality of doctors may independently transmit their doctor information and filter criteria. Transmission from each doctor may occur at different times. Each of these sets of doctor information and filter criteria is received at step 115. Each set of filter criteria is compared to the patient information at step 130. The patient information is then transmitted to each doctor to whom the patient information matches the filter criteria at step 130. A decision of willingness to schedule an appointment with the patient is received from a subset of doctors within the plurality of doctors at step 140. The size of this subset (i.e., the number of doctors in the subset) can be any number from one to the number of doctors in the plurality of doctors. The size of the subset may be set, for example, by the user so that the size of the subset is dependent on a certain number of doctors (for example, two or five or 10, etc.) the user desires to receive doctor information for at step 150. In some embodiments, the size of the subset will be the number of doctors that respond with a decision at step 140 between when the patient information is received and a length of time thereafter. This length of time may be set by the user (for example, one day, one week, two weeks, a month, etc.). In some embodiments, the size of the subset may be the number of doctors that respond until a number (e.g., one, five, ten, twenty, etc.) of doctors willing to schedule an appointment are found. In some embodiments, the user may select this number of doctors. In some embodiments, the patient information may comprise information relating to the size of the subset (e.g., the number of doctors willing to schedule an appointment with the patient, the length of time to find doctors willing to schedule an appointment, etc.).

The doctor information for each doctor willing to schedule an appointment is sent to the patient at step 150. Each set of doctor information may be sent as it is received or sets of doctor information may be sent together. At step 160, information specific to the geographic location proximal to the practice location of each doctor willing to schedule an appointment with the patient is sent to the patient. The user selects which doctors they would like to schedule an appointment with and receipt of that selection occurs at step 170. The doctor's contact information for each doctor selected by the patient and/or the facilitation of appointment making is then sent to the patient and/or user at step 200.

In some embodiments, patient filter criteria may be received from the user which dictates which doctors the patient information is transmit to at step 130. In some embodiments, patient filter criteria be received which dictates which doctor information or contact information is transmitted at step 150. In some embodiments, the patient information is sent to the doctor if the patient information matches the filter criteria and the doctor information matches the patient filter criteria. In some embodiments, the doctor information is sent to the patient and/or user if the doctor information matches the patient filter criteria.

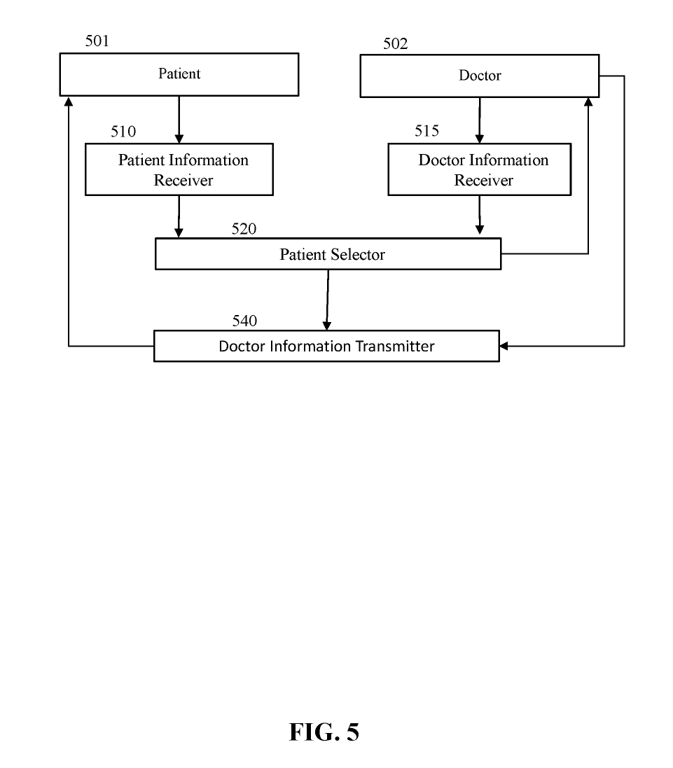

Referring now to FIG. 5, a system for arranging meetings between doctors and patients is disclosed. The system includes a patient information receiver, a doctor information receiver, a patient selector, and a doctor information transmitter. Patient Information Receiver 510 receives, preferably over a computer network, a cellular network, or the like, or through direct input at a local computer, patient information for one or more patients. In some embodiments, Patient Information Receiver 510 receives patient information by email, text message, instant messaging or the like. In some embodiments, Patient Information Receiver 510 receives patient information through a graphic user interface, such as a web page or software application accessible on a portable device, terminal or computer. In some embodiments, the patient information may include an identification of a medical need. In some embodiments, the patient information may be transmitted to Patient Information Receiver 510 by a patient, a patient's agent, a guardian, a referring doctor, an insurance company, a patient group, a government agency or the like. Doctor Information Receiver 515 receives, preferably over a computer network, a cellular network, or the like, or through direct input at a local computer, doctor information and/or filter criteria for one or more doctors. In some embodiments, Doctor Information Receiver 515 receives patient information by email, text message, instant messaging or the like. In some embodiments, Doctor Information Receiver 515 receives doctor information through a graphic user interface, such as a web page or software application accessible on a portable device, terminal or computer. In some embodiments, the doctor information and/or filter criteria for one or more doctors may be transmitted to Doctor Information Receiver 515 by a doctor, a doctor's agent, a health care provider group, or the like.

Patient Selector 520 receives patient information for one or more patients and doctor information and filter criteria for one or more doctors. In some embodiments, Patient Selector 520 compares the patient information for each patient with the filter criteria for each doctor, and if there is a match, Patient Selector 520 transmits the patient information for the matching patient to the matching doctor. In some embodiments, a set of filter criteria may require that a patient satisfy a subset of filter criteria for a match to be detected. In some embodiments, Patient Selector 520 collects patient information for one or more patients and transmits it to a matching doctor at the same time.

Doctor Information Transmitter 540 receives a decision of willingness to schedule an appointment with a patient from one or more matching doctors and transmits, preferably over a computer network, a cellular network, or the like, the doctor information for at least one matching doctor to the patient. In some embodiments, the doctor information includes contact information for the doctor. In some embodiments, Doctor Information Transmitter 540 transmits doctor information to the patient by email, text message, instant messaging or the like. In some embodiments, Doctor Information Transmitter 540 transmits doctor information to the patient through a graphic user interface, such as a web page or software application accessible on a portable device, terminal or computer. In some embodiments, Doctor Information Transmitter 540 receives appointment availability information from the one or more matching doctors. In some embodiments, Doctor Information Transmitter 540 may limit the sets of doctor information transmitted to a patient based upon a threshold. In some embodiments, the threshold may be defined by a patient or the transmitter of the patient information. In some embodiments, the threshold may be pre-defined by the system. In some embodiments, the number of sets of doctor information transmitted to a patient may be limited based upon the appointment availability of the corresponding doctor. In some embodiments, the threshold may relate to the number of doctors matched. In some embodiments, information specific to the geographic location proximal to the practice location of each doctor willing to schedule an appointment with the patient is sent to the patient. In some embodiments, the patient, the patient's agent, the patient's guardian, the referring doctor, the insurance company, the patient group, the government agency and/or the like selects which doctors they would like to schedule an appointment with for the patient, and the contact information for each doctor selected and/or the facilitation of appointment making is then sent to the patient, the patient's agent, the patient's guardian, the referring doctor, the insurance company, the patient group, the government agency and/or the like.

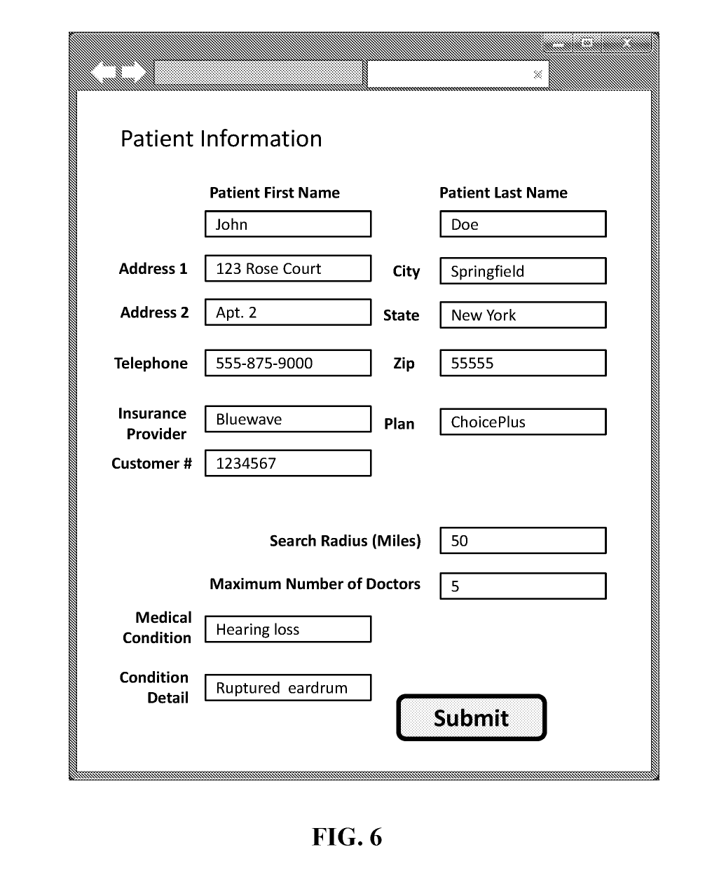

FIG. 6 shows an embodiment of a graphical user interface (“GUI”) for inputting patient information, which is transmitted to and received by Patient Information Receiver 510.

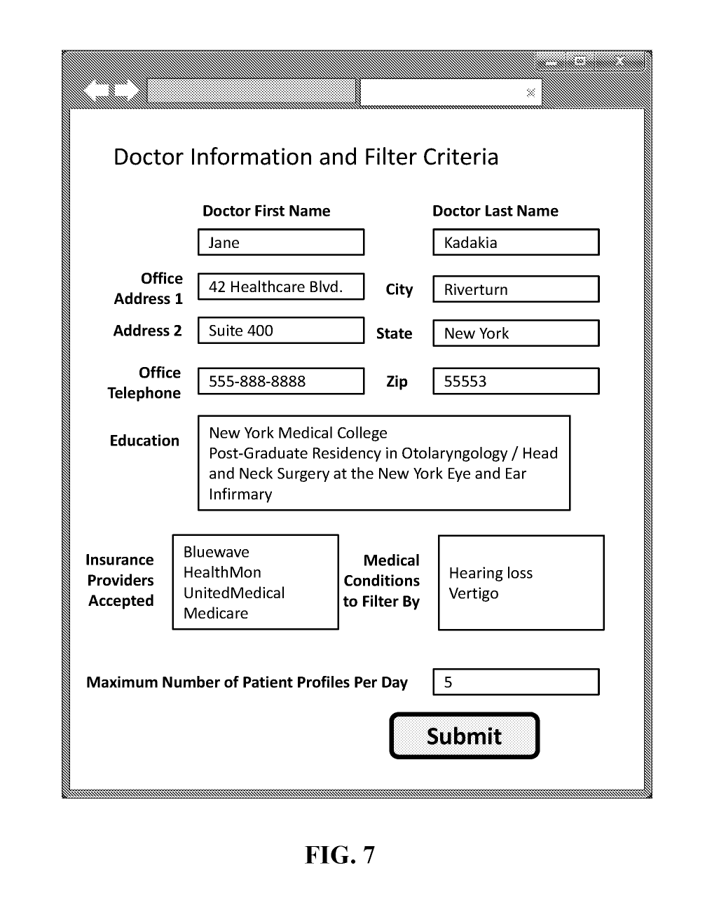

FIG. 7 shows an embodiment of a graphical user interface for inputting doctor information, which is transmitted to and received by Doctor Information Receiver 515.

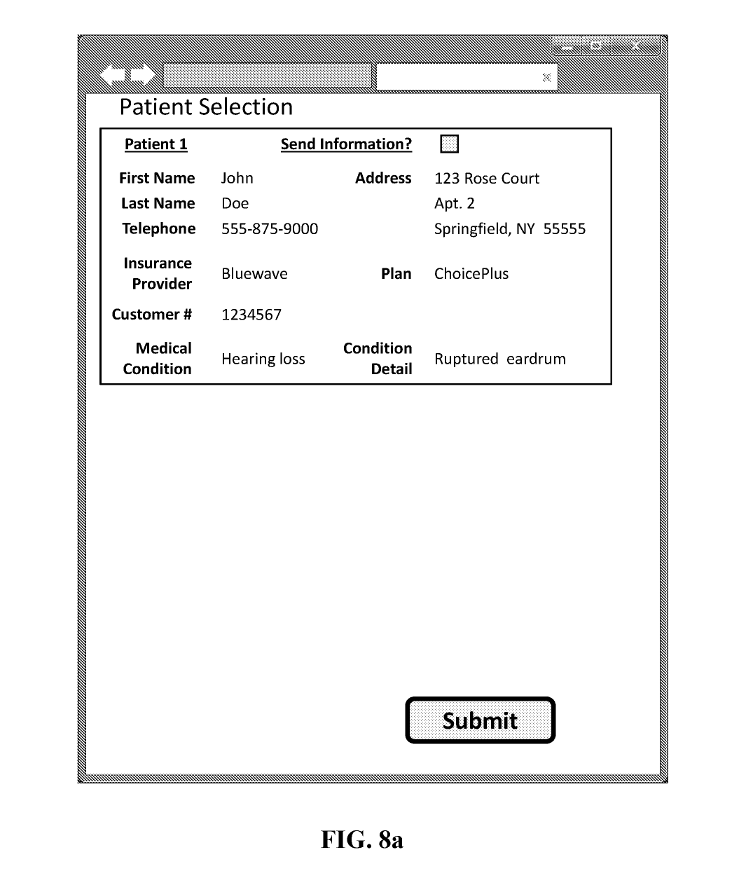

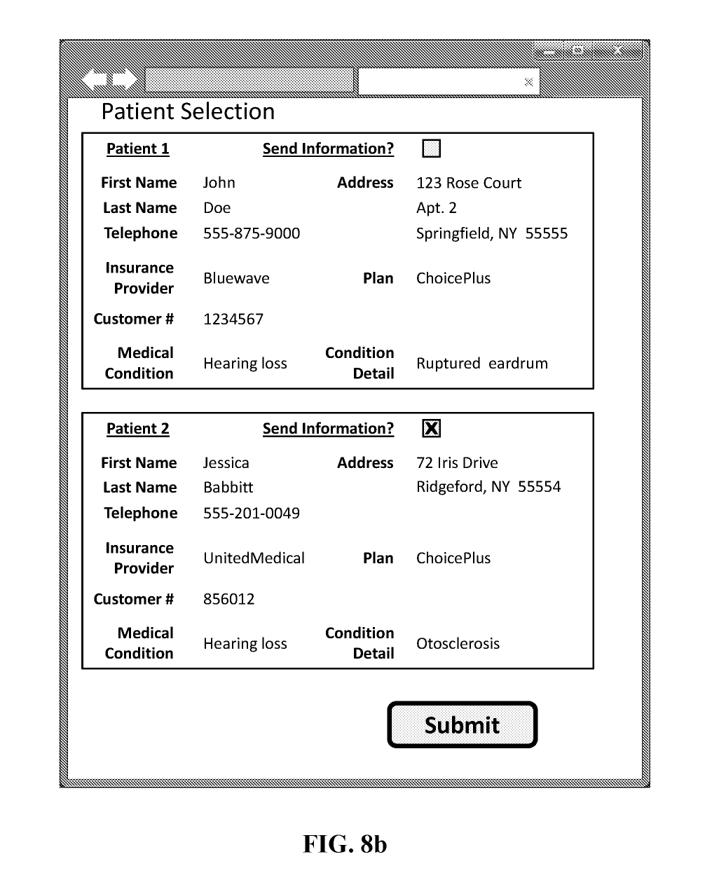

FIG. 8a shows an embodiment of a graphical user interface for selecting whether to send doctor information to a patient whose patient information was transmitted to a doctor via Patient Selector 520.

FIG. 8a shows an embodiment of a graphical user interface for selecting one or more patients to send doctor information to based on the patients whose patient information was transmitted to a doctor via Patient Selector 520.

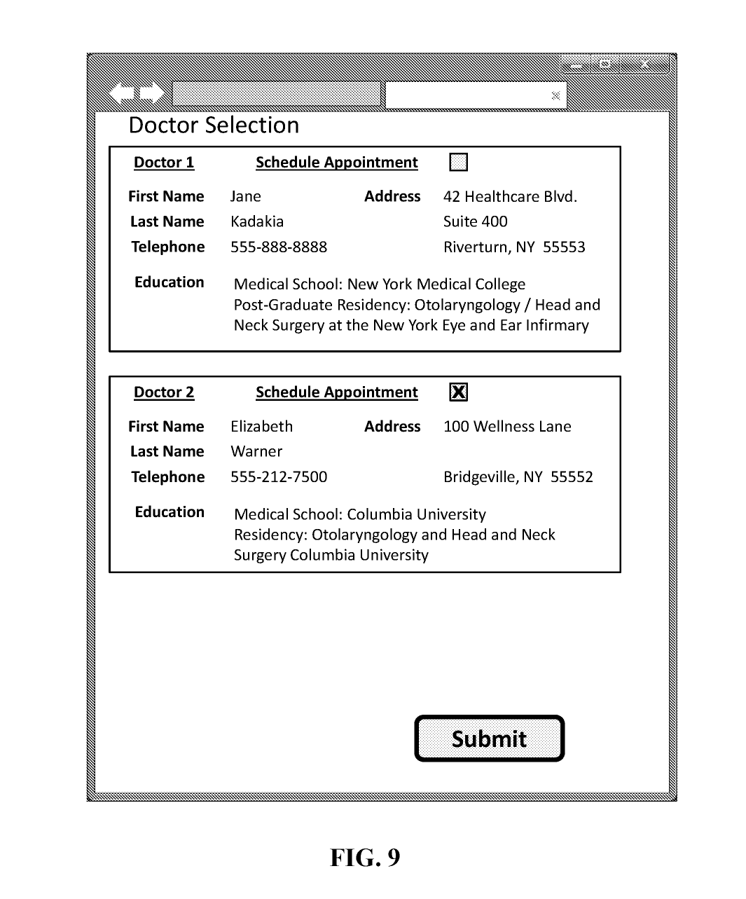

FIG. 9 shows an embodiment of a graphical user interface for scheduling an appointment with one or more doctors based on the doctor information transmitted to the patient, the patient's agent, the patient's guardian, the referring doctor, the insurance company, the patient group, the government agency and/or the like via Doctor Information Transmitter 540.

In one embodiment, the GUI's of FIGS. 6-9 may be implemented as web pages. In another embodiment, the GUI's of FIGS. 6-9 may be implemented as one or more applications which may be executed on a computer or portable device. In another embodiment, the GUI's of FIGS. 6-9 may be implemented as both web pages and one or more applications which may be executed on a computer or portable device. In one embodiment, one application and/or webpage is used by patients and another application and/or webpage is used by doctors.

While the principles of the disclosure have been described above in connection with specific methods and systems, it is to be understood that this description is made only by way of example and not as limitation on the scope of the disclosure.

1. A method of connecting a potential patient with a doctor comprising:
a. receiving, over a computer network, patient information from a user including an identification of a medical need;
b. receiving, over a computer network, filter criteria and doctor information from said doctor;
c. determining whether a match exists where said match occurs when said patient information matches said filter criteria;
d. transmitting, over a computer network, to said doctor, said patient information if said match exists;
e. receiving, over a computer network, a decision from said doctor indicating whether said doctor is willing to schedule an appointment with said user;
f. transmitting, over a computer network, to said user contact information of said doctor if said doctor is willing to schedule an appointment with said user.

2. The method according to claim 1 further comprising repeating the steps (a)-(h) with at least one other potential patient.

3. The method according to claim 2, wherein the patient information from said at least one other potential patient is transmitted to said doctor after receiving said decision from said doctor.

4. The method according to claim 1 further comprising:
receiving, over a computer network doctor information and filter criteria from at least one other doctor; and
transmitting, over a computer network, said doctor information for each doctor that is willing to schedule an appointment with said user.

5. The method of claim 1 further comprising receiving available appointment times from said doctor if said doctor is willing to schedule an appointment.

6. The method of claim 1 further comprising scheduling an appointment for said potential patient with said doctor.

7. The method of claim 1 further comprising receiving, over a computer network, referral information for one or more other practitioners from said doctor if said doctor is unwilling to schedule an appointment.

8. The method of claim 1 further comprising transmitting, over a computer network, to said user information specific to a geographic location proximal with the practice location of said doctor.

9. The method according to claim 8, wherein said information is advertising specific to said geographic location.

10. The method according to claim 9, wherein said advertising relates to amenities.

11. The method according to claim 10, wherein said amenities are selected from the group consisting of lodging, dining, food delivery, transportation, and entertainment.

12. The method according to claim 10, wherein said amenities are selected from the group consisting of pharmacies, nursing services, and rehabilitation services.

13. The method of claim 1 further comprising transmitting, over a computer network, patient evaluations relating to said doctor, where said patient evaluations are collected from previous patients of said doctor.

14. The method of claim 13, wherein said patient evaluations comprise a rating system.

15. The method of claim 14, wherein said rating system is numeric.

16. The method of claim 1, wherein said doctor information comprises data about said doctor's experience, credentials, and/or awards.

17. A system for connecting a potential patient with a doctor comprising:
a. patient information receiver for receiving patient information from a user including an identification of a medical need;
b. doctor information receiver for receiving filter criteria and doctor information including contact information from said doctor;
c. patient information analyzer for generating a match indication when said patient information matches said filter criteria;
d. patient selector for comparing said patient information with said filter criteria to generate a match indicator and transmitting said patient information to said doctor based on said match indicator;
e. doctor information transmitter for receiving a decision from said doctor indicating whether said doctor is willing to schedule an appointment with said user and transmitting to said user said doctor information if said doctor is willing to schedule an appointment with said user.
